(19) United States
(12) Patent Application Publication
(10) Pub. No.: US 2020/0209213 A1
(43) Pub. Date: Jul. 2, 2020

(54) METHOD FOR DETERMINING THE COMPOSITION AND PROPERTIES OF HYDROCARBON FRACTIONS BY SPECTROSCOPY OR SPECTROMETRY

(71) Applicant: Saudi Arabian Oil Company, Dhahran (SA)

(72) Inventors: Omer Refa Koseoglu, Dhahran (SA); Tulay Y. Inan, Dhahran (SA)

(21) Appl. No.: 16/233,562

(22) Filed: Dec. 27, 2018

(51) Int. Cl. G01N 33/28; G01J 3/28; G01N 21/359; G01N 21/3577; C10G 7/00

(52) U.S. Cl. CPC G01N 33/2829; G01J 3/28; G01N 2021/3595; G01N 21/3577; C10G 7/00; G01N 21/359

(57) ABSTRACT

This invention relates to a system and method for the evaluation of samples of a distillate fraction by spectroscopic analysis, followed by the application of chemometrics software to determine physical characteristics of the fraction.

### FIELD OF THE INVENTION

This invention relates to a system and method for the evaluation of samples of a distillate fraction by spectroscopic analysis, followed by the application of chemometrics software to determine physical characteristics of the fraction.

### BACKGROUND OF THE INVENTION

Crude oil originates from the decomposition and transformation of aquatic, mainly marine, living organisms and/or land plants that became buried under successive layers of mud and silt some 15-500 million years ago. They are essentially very complex mixtures of many thousands of different hydrocarbons. Depending on the source, the oil predominantly contains various proportions of straight and branched-chain paraffins, cycloparaffins, and naphthenic, aromatic, and polynuclear aromatic hydrocarbons. These hydrocarbons can be gaseous, liquid, or solid under normal conditions of temperature and pressure, depending on the number and arrangement of carbon atoms in the molecules.

Crude oils vary widely in their physical and chemical properties from one geographical region to another and from field to field. Crude oils are usually classified into three groups according to the nature of the hydrocarbons they contain: paraffinic, naphthenic, asphaltic, and their mixtures. The differences are due to the different proportions of the various molecular types and sizes. One crude oil can contain mostly paraffins, another mostly naphthenes. Whether paraffinic or naphthenic, one can contain a large quantity of lighter hydrocarbons and be mobile or contain dissolved gases; another can consist mainly of heavier hydrocarbons and be highly viscous, with little or no dissolved gas. Crude oils can also include heteroatoms containing sulfur, nitrogen, nickel, vanadium and other elements in quantities that impact the refinery processing of the crude oil fractions. Light crude oils or condensates can contain sulfur in concentrations as low as 0.01 W %; in contrast, heavy crude oils can contain as much as 5-6 W %. Similarly, the nitrogen content of crude oils can range from 0.001-1.0 W %.

The nature of the crude oil governs, to a certain extent, the nature of the products that can be manufactured from it and their suitability for special applications. A naphthenic crude oil will be more suitable for the production of asphaltic bitumen, a paraffinic crude oil for wax. A naphthenic crude oil, and even more so an aromatic one, will yield lubricating oils with viscosities that are sensitive to temperature. However, with modern refining methods there is greater flexibility in the use of various crude oils to produce many desired type of products.

A crude oil assay is a traditional method of determining the nature of crude oils for benchmarking purposes. Crude oils are subjected to true boiling point (TBP) distillations and fractionations to provide different boiling point fractions. The crude oil distillations are carried out using the American Standard Testing Association (ASTM) Method D 2892. The common fractions and their nominal boiling points are given in Table 1.

TABLE 1

| | Boiling Point, |
|---|---|
| Fraction | ° C. |
| Methane | −161.5 |
| Ethane | −88.6 |
| Propane | −42.1 |
| Butanes | −6.0 |
| Light Naphtha | 36-90 |
| Mid Naphtha | 90-160 |
| Heavy Naphtha | 160-205 |
| Light gas Oil | 205-260 |
| Mid Gas Oil | 260-315 |
| Heavy gas Oil | 315-370 |
| Light Vacuum Gas Oil | 370-430 |
| Mid Vacuum Gas Oil | 430-480 |
| Heavy vacuum gas oil | 480-565 |
| Vacuum Residue | 565+ |

The yields, composition, physical and indicative properties of these crude oil fractions, where applicable, are then determined during the crude assay work-up calculations. Typical compositional and property information obtained from a crude oil assay is given in Table 2.

TABLE 2

| | | Property | |
|---|---|---|---|
| Property | Unit | Type | Fraction |
| Yield Weight and | W % | Yield | All |
| Volume % | | | |
| API Gravity | ° | Physical | All |
| Viscosity Kinematic | ° | Physical | Fraction boiling >250° C. |
| @ 38° C. | | | |
| Refractive Index | Unitless | Physical | Fraction boiling <400° C. |
| @ 20° C. | | | |
| Sulfur | W % | Composition | All |
| Mercaptan Sulfur, | W % | Composition | Fraction boiling <250° C. |
| W % | | | |
| Nickel | ppmw | Composition | Fraction boiling >400° C. |
| Nitrogen | ppmw | Composition | All |
| Flash Point, COC | ° C. | Indicative | All |
| Cloud Point | ° C. | Indicative | Fraction boiling >250° C. |
| Pour Point, (Upper) | ° C. | Indicative | Fraction boiling >250° C. |
| Freezing Point | ° C. | Indicative | Fraction boiling >250° C. |
| Micro Carbon Residue | W % | Indicative | Fraction boiling >300° C. |
| Smoke Point, mm | mm | Indicative | Fraction boiling between |
| | | | 150-250° C. |
| Octane Number | Unitless | Indicative | Fraction boiling <250° C. |
| Cetane Index | Unitless | Indicative | Fraction boiling between |
| | | | 150-400° C. |
| Aniline Point | ° C. | Indicative | Fraction boiling <520° C. |

Due to the number of analyses involved, the oil assay is both costly and time consuming.

In a typical refinery, crude oil is first fractionated in the atmospheric distillation column to separate sour gas and light hydrocarbons, including methane, ethane, propane, butanes and hydrogen sulfide, naphtha (36-180° C.), kerosene (180-240° C.), gas oil (240-370° C.) and atmospheric residue (>370° C.). The atmospheric residue from the atmospheric distillation column is either used as fuel oil or sent to a vacuum distillation unit, depending on the configuration of the refinery. The principal products obtained from vacuum distillation are vacuum gas oil, comprising hydrocarbons boiling in the range 370-520° C., and vacuum residue, comprising hydrocarbons boiling above 520° C. Crude assay data is conventionally obtained from individual analysis of these cuts to help refiners to understand the general composition of the crude oil fractions and properties so that the fractions can be processed most efficiently and effectively in an appropriate refining unit. Indicative properties are used to determine the engine/fuel performance or usability or flow characteristic or composition. A summary of the indicative properties and their determination methods with description is given below.

The cetane number of diesel fuel oil, determined by the ASTM D613 method, provides a measure of the ignition quality of diesel fuel; as determined in a standard single cylinder test engine; which measures ignition delay compared to primary reference fuels. The higher the cetane number; the easier the high-speed; direct-injection engine will start; and the less white smoking and diesel knock after start-up are. The cetane number of a diesel fuel oil is determined by comparing its combustion characteristics in a test engine with those for blends of reference fuels of known cetane number under standard operating conditions. This is accomplished using the bracketing hand wheel procedure which varies the compression ratio (hand wheel reading) for the sample and each of the two bracketing reference fuels to obtain a specific ignition delay, thus permitting interpolation of cetane number in terms of hand wheel reading.

The cloud point, determined by the ASTM D2500 method, is the temperature at which a cloud of wax crystals appears when a lubricant or distillate fuel is cooled under standard conditions. Cloud point indicates the tendency of the material to plug filters or small orifices under cold weather conditions. The specimen is cooled at a specified rate and examined periodically. The temperature at which cloud is first observed at the bottom of the test jar is recorded as the cloud point. This test method covers only petroleum products and biodiesel fuels that are transparent in 40 mm thick layers, and with a cloud point below 49° C.

The pour point of petroleum products, determined by the ASTM D97 method, is an indicator of the ability of oil or distillate fuel to flow at cold operating temperatures. It is the lowest temperature at which the fluid will flow when cooled under prescribed conditions. After preliminary heating, the sample is cooled at a specified rate and examined at intervals of 3° C. for flow characteristics. The lowest temperature at which movement of the specimen is observed is recorded as the pour point.

The aniline point, determined by the ASTM D611 method, is the lowest temperature at which equal volumes of aniline and hydrocarbon fuel or lubricant base stock are completely miscible. A measure of the aromatic content of a hydrocarbon blend is used to predict the solvency of a base stock or the cetane number of a distillate fuel. Specified volumes of aniline and sample, or aniline and sample plus n-heptane, are placed in a tube and mixed mechanically. The mixture is heated at a controlled rate until the two phases become miscible. The mixture is then cooled at a controlled rate and the temperature at which two separate phases are again formed is recorded as the aniline point or mixed aniline point.

The octane number, determined by the ASTM D2699 or D2700 methods, is a measure of a fuel's ability to prevent detonation in a spark ignition engine. Measured in a standard single-cylinder; variable-compression-ratio engine by comparison with primary reference fuels. Under mild conditions, the engine measures research octane number (RON), while under severe conditions, the engine measures motor octane number (MON). Where the law requires posting of octane numbers on dispensing pumps, the antiknock index (AKI) is used. This is the arithmetic average of RON and MON, (R+M)/2. It approximates the road octane number, which is a measure of how an average car responds to the fuel.

To determine these properties of gas oil or naphtha fractions conventionally, these fractions have to be distilled from the crude oil and then measured/identified using various analytical methods that are laborious, costly and time-consuming.

This invention is directed to a refinery that has already fractionated a distillate, such as diesel, and seeks to determine its composition and properties.

ASTM D2425, “Standard Test Method for Hydrocarbon Types in Middle Distillates by Mass Spectrometry,” can be used to identify the hydrocarbon types present in virgin middle distillates in the 204-343° C. (400-650° F.) boiling range, 5% to 95% by volume as determined by Test Method D86. Samples with average carbon number value of paraffins between C₁₂ and C₁₆ and containing paraffins from C₁₀ and C₁₈ can be analyzed. A number of hydrocarbon types can be determined, including: paraffins, noncondensed cycloparaffins, condensed dicycloparaffins, condensed tricycloparaffins, alkylbenzenes, indans or tetralins, or both, CₙH₂ₙ₋₁₀ (indenes, etc.), naphthalenes, CₙH₂ₙ₋₁₄ (acenaphthenes, etc.), CₙH₂ₙ₋₁₆ (acenaphthylenes, etc.), and tricyclic aromatics.

ASTM D1218, “Standard Test Method for Refractive Index and Refractive Dispersion of Hydrocarbon Liquids,” can be used to determine the refractive indices of transparent and light-colored hydrocarbons in the range of 1.3300 to 1.5000 at temperatures from 20-30° C. liquids. The measurement is accurate and reported to four decimal points.

ASTM D4052, “Standard Test Method for Density, Relative Density, and API Gravity of Liquids by Digital Density Meter,” is used to determine the density, relative density, and API Gravity of petroleum distillates and viscous oils that can be handled in a normal fashion as liquids at the temperature of test, utilizing either manual or automated sample injection equipment. Its application is restricted to liquids with total vapor pressures (see Test Method D5191) typically below 100 kPa and viscosities (see Test Method D445 or D7042) typically below about 15 000 mm2/s at the temperature of test. The total vapor pressure limitation however can be extended to >100 kPa provided that it is first ascertained that no bubbles form in the U-shaped, oscillating tube, which can affect the density determination. Some examples of products that may be tested by this procedure include: gasoline and gasoline-oxygenate blends, diesel, jet, basestocks, waxes, and lubricating oils.

ASTM D5291, “Standard Test Methods for Instrumental Determination of Carbon, Hydrogen, and Nitrogen in Petroleum Products and Lubricants,” covers the instrumental determination of carbon, hydrogen, and nitrogen in laboratory samples of petroleum products and lubricants. Values obtained represent the total carbon, the total hydrogen, and the total nitrogen.

Chemometrics, or multivariate data analysis, describes optimal mathematical and statistical methods to process large amounts of data. It enables users to design experiments and analyze the data generated to get valuable information after measurements have been taken. The need for chemometrics tools mainly comes from the development of analytical instruments providing large amounts of complex data. Although chemometrics is based in mathematics and statistics, one does not need to have deep knowledge to analyze multivariate data. However, thorough knowledge of the application as well as common sense are required in order to analyze the data generated to avoid misinterpretations.

The conventional standard methods require a large amount of a sample, consume time, and may involve the use of toxic or environmentally dangerous reagents. In experimental analytical chemistry, an interdisciplinary technique of chemometrics has been widely used to determine patterns and relationships. It uses mathematical methods and algorithms for the analysis of results to predict the physical and/or chemical properties of the analyzed samples. For analyzing chemical systems, including fuels, multivariate calibration has been used as one application of chemometrics. For example, Fourier-transform infrared spectroscopy (FT-IR) spectroscopy is a nondestructive, rapid, and easy analytical method that is based on the measurement of characteristic fundamental resonances for different functional groups. It produces well-defined peaks at wavelengths between 2.5-25 μm, corresponding to the 4000-650 cm⁻¹ wavenumber region. Use of FT-IR spectroscopy to determine diesel and gasoline fuel properties has been extensive.

Although these methods are available to predict the properties of the oil directly from the spectrum of the oil sample, they lack accuracy, within definition of the standard analytical method. So the inventors are disclosing a method to predict composition and properties of oil fractions using spectrometers, chemometrics and the newly developed method.

Predictive technology reduces the number of laboratory analyses conducted. This system and method allows refining and R&D pilot plant operations to predict the property in a cost- and time-effective manner with an analyzer, rather than through costly and lengthy measurements involving lab measurements and chemicals.

New rapid, and direct methods to help better understand the properties of distillates will save producers, marketers, refiners and/or other crude oil users substantial expense, effort and time.

### SUMMARY OF THE INVENTION

Systems and methods for determining at least one indicative property or composition of a hydrocarbon sample are presented. The hydrocarbon sample is a distillate fraction, such as diesel. The fraction is sampled with a spectroscope or spectrometer. Then, chemometrics software is applied to the spectroscopic data to determine physical characteristics, such as boiling point, paraffin content, naphthene content, aromatic content, hydrogen content, and carbon content.

### BRIEF DESCRIPTION OF THE DRAWINGS

Further advantages and features of the present invention will become apparent from the following detailed description of the invention when considered with reference to the accompanying drawings in which:

FIG. 1 is a process flow diagram of steps of a method in which an embodiment of the invention is implemented;

FIG. 2 is a schematic block diagram of modules of an embodiment of the invention; and

FIG. 3 is a block diagram of a computer system in which an embodiment of the invention is implemented.

### DETAILED DESCRIPTION OF INVENTION

A system and a method are provided for analyzing a distillate fraction to determine physical characteristics of the fraction. The fraction is first analyzed by a spectroscope or spectrometer. From the spectroscopic data, chemometrics software is employed to determine physical characteristics such as boiling point, paraffin content, naphthene content, aromatic content, hydrogen content, and carbon content.

In a refinery or pilot plant that processes a distillate fraction, such as diesel, a very small amount of the distillate fraction, such as 2 ml, is sampled. This sampling is preferably accomplished with an online, continuous measurement. Alternatively, an off-line measurement is used to obtain the sample.

The sample is then analyzed by a spectroscope or spectrometer, at least the following types of which are suitable: near infrared (NIR) spectrometer, Fourier transform infrared (FTIR) spectrometer, nuclear magnetic resonance (NMR) spectrometer, ultraviolet visible (UV-Vis) spectrometer, Fourier transform-ion cyclotron-mass spectrometer (FT-ICR MS), time-of-flight mass spectrometer (TOF-MS), laser inducted UV spectrometer, or fluorescence spectrometer.

In developing this invention, diesel samples from nine difference sources were distilled from different crude oils. A batch distiller was used to recover naphtha and diesel. The nominal diesel cut point range is 180-370° C. The properties of these samples were determined using the standard methods. Table 3 summarizes the composition and properties of one of these nine gas oils as an example.

TABLE 3

| Code | Unit | Method | 6880 |
|---|---|---|---|
| Density | g/cm3 | ASTM D4052 | 0.8423 |
| Refractive index @ 20 □ C. | — | ASTM D1218 | 1.470 |
| Viscosity @ 40 □ C. | cSt | ASTM D445 | 2.89 |
| Hydrogen | W % | ASTM D5291 | 13.21 |
| Carbon | W % | ASTM D5291 | 85.27 |
| Cetane Number | — | ASTM D613 | 60 |
| Paraffins | W % | ASTM D2425 | 44.5 |
| Naphthenes | W % | ASTM D2425 | 23.4 |
| Aromatics | W % | ASTM D2425 | 32.1 |
| Distillation | ° C. | | |
| 0 | W % | ASTM D2887 | 141 |
| 5 | W % | ASTM D2887 | 188 |
| 10 | W % | ASTM D2887 | 204 |
| 30 | W % | ASTM D2887 | 249 |
| 50 | W % | ASTM D2887 | 285 |
| 70 | W % | ASTM D2887 | 319 |
| 90 | W % | ASTM D2887 | 351 |
| 95 | W % | ASTM D2887 | 364 |
| 100 | W % | ASTM D2887 | 400 |
| 1H NMR-Aromatic proton | % | NMR | 4.57 |

Seven gas oil blends derived from various crude oils by true boiling point distillation were used to predict the paraffins, naphthenes and aromatics contents using an FTIR spectrometer and chemometrics. The FTIR spectrum was collected for each diesel sample that was previously analyzed according to the standard test methods as shown in Table 1, and the resulting spectroscopic data was analyzed by chemometrics to predict the composition of the oil samples. Table 4 tabulates the results for paraffins, naphthenes, and aromatics, showing the actual values (as determined by the ASTM methods), the predicted values (based upon the chemometric analysis of spectroscopic data), and average deviations for each. As seen, the concentration of the oil components can be predicted almost as accurately as measured by the ASTM method. The average absolute deviation (AAD) is calculated as shown in equation 1:

$$A A D = absolute [ \frac{( actual value - predicted value )}{actual value} * 100 ] ; \qquad ( 1 )$$

TABLE 4

| | Paraffins | | | Naphthenes | | | Aromatics | | |
|---|---|---|---|---|---|---|---|---|---|
| Code | Actual | Predicted | AAD | Actual | Predicted | AAD | Actual | Predicted | AAD |
| F-322 | 22.4 | 22.5 | 0.4 | 48.8 | 48.9 | 0.2 | 28.8 | 27.4 | 4.9 |
| F-328 | 38.7 | 38.5 | 0.4 | 33.1 | 33.1 | 0.0 | 28.2 | 28.0 | 0.7 |
| F-327 | 40.8 | 40.9 | 0.3 | 35.7 | 35.3 | 1.1 | 23.5 | 23.3 | 0.9 |
| F-320 | 44.5 | 44.7 | 0.5 | 23.4 | 23.5 | 0.4 | 32.1 | 32.4 | 0.9 |
| F-318 | 46.1 | 46.2 | 0.2 | 24.8 | 24.9 | 0.4 | 29.1 | 28.2 | 3.0 |
| F-324 | 47.4 | 47.2 | 0.5 | 24.4 | 24.5 | 0.4 | 28.2 | 28.6 | 1.4 |
| F-321 | 47.5 | 47.5 | 0.0 | 25.0 | 25.1 | 0.4 | 27.5 | 28.9 | 5.1 |
| Average | | | 0.3 | | | 0.4 | | | 2.4 |
| Reproducibility | | | 5 | | | 4 | | | 3 |

The paraffins, naphtenes and aromatics composition of the oil fractions were estimated with high accuracy, AAD of 0.3 W %, 0.4 W % and 2.4 W %, respectively. The average values are well below the method's reproducibility values except that of aromatics. The average AAD for aromatics was 2.4% and the reproducibility for the method is 1.4 W %.

The correlation is performed against the spectral data through statistical analysis or trends, using statistical methods such as classical least-squares (CLS), inverse least squares (ILS), principal-component regression (PCR), artificial neural network (ANN), partial least-squares (PLS) and net-analyte signal (NAS). When a spectrometer other than the cited FTIR spectrometer is used, the spectra are different, but the same chemometrics methods can be used.

In this example, the Partial Least Squares (PLS) regression was used to correlate the spectroscopic data to middle distillate (MD) property values (for each property). The PLS algorithm was run from Nicolets's TQ Anlayst Software package.

The PLS method creates a simplified representation of the spectroscopic data by a process known as spectral decomposition.

The PLS algorithm initially calculates a property value (naphthenes, aromatics . . . etc.), or weighted average spectrum of all spectra of the MDs in the calibration matrix.

This statistical analysis requires calibration and validation.

In the calibration procedure, the software searches for a relation between the dependent variable, Y (peak height), and the independent variable, X (property) which can be generically written as: Y=f(X1, X2, X3 . . . Xp).

In practice, an algorithm, based on PLS, calculates the regression coefficients of the following equation:

Y=b0+b1X1+b2X2+ . . . bpXp (2);

This defines the mathematical model of the system under investigation. The second step is a so-called “leave-one-out” cross-validation procedure that is used to verify the calibration model.

FTIR and multivariable calibration methods accuracy was established by evaluating Root Mean Square Error of Prediction (RMSEP), Root Mean Square Error of Calibration (RMSEC) and Correlation coefficient (R²), and after cross-validation, Root Mean Square Error of Cross Validated error of calibration (RMSECV) and the correlation coefficient (R²) added as statistical evaluation parameters

These calculations have been done by the software.

FTIR and multivariable calibration methods accuracy was established by evaluating the root mean square error of prediction (RMSEP), the root mean square error of calibration (RMSEC) and the correlation coefficient (R²) and after cross-validation, cross validated error of calibration (RMSECV) and the correlation coefficient (R²) added as statistical evaluation parameters.

It is critical to establish the correct number of factors to be used in the correlation files, as the predicted MD property values calculated from the model depend on the number of factors used in the model. Too few factors will not adequately model the system, while too many factors will introduce noise vectors in the calibration. These noise vectors will result in less than optimum prediction for samples outside the calibration set. The Nicolet TQ Analyst program provides RMSECV data by plotting the Predicted Residual Error Sum of Squares (PRESS, which is a factor analysis method) versus model factors (1-10) to select the appropriate factor.

Predicted Residual Error Sum of Squares, PRESS measures how well the calibration model predicts the property value as each factor is added, defined as:

$$PRESS = {\sum }_{i = 1}^{n} {( {y}_{i} - {\hat{y}}_{i} )}^{2} = {\sum }_{i = 1}^{n} {e}_{i}^{2} ; \qquad ( 3 )$$

Where yᵢ is the actual value of y for object i and the y-value ŷᵢ for object i predicted with the model under evaluation, is the residual for object i (the y difference between the predicted and the actual y-value) and n is the number of objects for ŷ which I is obtained by prediction. The PRESS graph produced by TQ analyst software.

The mean squared error of prediction (MSEP) is defined as the mean value of PRESS:

$$MSEP = \frac{PRESS}{n} = \frac{{\sum }_{i = 1}^{n} {( {y}_{i} - {\hat{y}}_{i} )}^{2}}{n} = \frac{{\sum }_{i = 1}^{2} {e}_{i}^{2}}{n} \qquad ( 4 )$$

Root mean squared error of prediction (RMSEP) is the MSEPs square root:

$$RMSEP = \sqrt{MSEP} = \sqrt{\frac{{\sum }_{i = 1}^{n} {( {y}_{i} - {\hat{y}}_{i} )}^{2}}{n}} = \sqrt{\frac{{\sum }_{i = 1}^{n} {e}_{i}^{2}}{n}} \qquad ( 5 )$$

In the chemometrics literature, it seems that RMSEP values are preferred, partly because they are given in the same units as the y-variable.

Root Mean Standard Error of Calibration for Cross Validation, RMSECV the cross-validation process accompanies the construction of the “full” model, which uses the complete set of the loaded data. Consequently, once the analysis is executed, the cross-validation results and plots will be made available along with the results and the plots for the “full” model. For example, the model results window in the analysis will include the Root Mean Square Error of Cross-Validation (RMSECV) value along with the Root Mean Square Error of Calibration (RMSEC) value. The RMSECV is defined as

$$RMSECV = \sqrt{\frac{{\sum }_{i = 1}^{n} {( {y}_{i} - {\hat{y}}_{i} )}^{2}}{n}} \qquad ( 6 )$$

Where ŷ contains the values of the Y variable that are estimated by cross-validation (where the value for each object i is estimated using a model that was built using a set of objects that does not include object i), y contain the known values of the Y variable, and n is the total number of objects in the data set.

Square of the Multiple Correlation Coefficient, R², The multiple correlation coefficient, R², is a measure of how well a linear model fits a given set of data, and has a value between 0 and 1. If the estimated and known values are very similar, then a good fit is achieved and R² will be close to 1. A poor fit will give an R² closer to 0. R² can be interpreted as the total variability in y (MD property values) that is explained by x (spectral data). However, caution must be taken in using R² because a large value for R² does not necessarily mean a good fit between the model and the data. In Equation 7, for a given MD property of sample yᵢ, we have the known value from the standard method (ASTM) and the yᵢ′ (predicted value by the model) and y″ (the mean of all yᵢ), and n (the number of samples).

$${R}^{2} = 1 - \frac{{\sum }_{i = 1}^{∞} {( {y}_{i} - {y}_{i}^{′} )}^{2}}{{\sum }_{i = 1}^{∞} {( {y}_{i} - {y}_{i}^{″} )}^{2}} ; \qquad ( 7 )$$

Principal component analysis (PCA) is a statistical procedure and PCA is sensitive to the relative scaling of the original variables. PCA is the simplest of the true eigenvector-based multivariate analyses. Often, its operation can be thought of as revealing the internal structure of the data in a way that best explains the variance in the data. By using this method you can observe unknown samples behaviors/distribution in multivariate analysis and these calculations had been done for this work using the mentioned software.

Finally, additional physical properties and compositional values of the distillate fraction are estimated by correlations as a function of at least two of the three parameters of paraffin, naphthene, and aromatic content. Table 5 shows the AAD for density, cetane number, hydrogen, aromatic hydrogen determined by NMR, viscosity at 40° C., refractive index, and distillation 50 W % point. As seen, the method predicts composition and properties of the oil fractions accurately. Note that aromatic hydrogen determined by NMR is different from the aromatic composition. The aromatic composition refers to the whole aromatic molecule, whereas the aromatic hydrogen refers only to the hydrogen bonded to the aromatic rings.

TABLE 5

| | | | | | | | Dist. |
|---|---|---|---|---|---|---|---|
| | | Cetane | | Aromatic | Viscosity | Refractive | 50 W % |
| S# | Density | Number | Hydrogen | Hydrogen | @40° C. | Index | Point |
| 318 | 0.05 | 0.29 | 0.11 | 1.13 | 2.24 | 0.02 | 0.42 |
| 320 | 0.01 | 0.00 | 0.03 | 0.11 | 0.48 | 0.01 | 0.06 |
| 321 | 0.04 | 0.30 | 0.01 | 0.74 | 0.46 | 0.01 | 0.21 |
| 322 | 0.01 | 0.02 | 0.01 | 0.01 | 0.04 | 0.00 | 0.00 |
| 323 | 0.17 | 0.21 | 0.20 | 0.21 | 2.25 | 0.05 | 0.20 |
| 324 | 0.04 | 0.57 | 0.03 | 1.59 | 1.63 | 0.00 | 0.49 |
| 325 | 0.01 | 0.00 | 0.00 | 0.00 | 0.01 | 0.01 | 0.00 |
| 327 | 0.02 | 0.02 | 0.02 | 0.03 | 0.28 | 0.01 | 0.03 |
| 328 | 0.13 | 0.22 | 0.15 | 0.02 | 1.44 | 0.04 | 0.09 |
| Average | 0.05 | 0.18 | 0.06 | 0.43 | 0.98 | 0.14 | 1.51 |

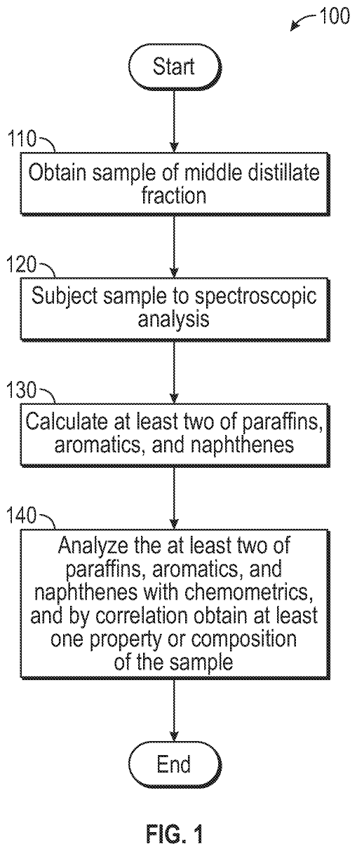

FIG. 1 shows a process flowchart of steps in a method according to one embodiment herein. In step 110, a sample of approximately 2 ml of a distillate fraction is obtained. This sampling is preferably accomplished with an online, continuous measurement. Alternatively, an off-line measurement is used to obtain the sample.

In step 120, the sample is subjected to spectroscopic analysis by a spectrometer, such as a near infrared (NIR) spectrometer, Fourier transform infrared (FTIR) spectrometer, nuclear magnetic resonance (NMR) spectrometer, ultraviolet visible (UV-Vis) spectrometer, Fourier transform-ion cyclotron-mass spectrometer (FT-ICR MS), time-of-flight mass spectrometer (TOF-MS), laser inducted UV spectrometer, or fluorescence spectrometer. The spectroscopic data is stored into a non-volatile memory device.

In a preferred embodiment, the spectrometer is a near infrared (NIR) spectrometer, and the wavenumber of the spectrum is in the range of 4,000-12,821 cm⁻¹.

In a preferred embodiment, the spectrometer is a Fourier transform infrared (FTIR) spectrometer, and the wavelength is in the range of 650-2000 cm⁻¹.

In a preferred embodiment, the spectrometer is a nuclear magnetic resonance (NMR) spectrometer, that is carbon- or hydrogen-based.

In a preferred embodiment, the spectrometer is an ultraviolet visible (UV-Vis) spectrometer, and the wavelength is in the range of 220-900 nm.

In a preferred embodiment, the spectrometer is a Fourier transform-ion cyclotron-mass spectrometer (FT-ICR MS), covering masses in the range of 150-1400 m/z.

In a preferred embodiment, the spectrometer is a Fourier transform-ion cyclotron-mass spectrometer (FT-ICR MS), covering carbon numbers in the range 1-60.

In a preferred embodiment, the spectrometer is a time-of-flight mass spectrometer (TOF-MS), operating at 5-20 kHz repetition rates.

In a preferred embodiment, the spectrometer is a fluorescence spectrometer, with a wavelength in the range of 250-800 nm.

In step 130, at least two compositional variable parameters of the fraction are predicted from the results of the spectroscopic analysis, with the at least two compositional variable parameters being selected from the paraffins, naphthenes, aromatics. These at least two compositional variable parameters are stored into the non-volatile memory device. As discussed earlier, in one example, several samples that were previously analyzed according to standard test methods were then analyzed by FTIR spectrometer, and the resulting data were then used to establish calibration models with the corresponding compositional variable parameters obtained from the standard methods. In that way, use of an FTIR spectrometer and chemometrics on an unknown sample can be used to predict the composition of the unknown sample.

Multivariate calibration is an effective calibration method in which the chemical information, such as absorption, emission, transmission, etc., of a set of standard mixtures recorded at different variables (wavenumbers) are related to the concentration of the chemical compounds present in the mixtures. The correlation is performed against the spectral data through statistical analysis or trends, using statistical methods such as classical least-squares (CLS), inverse least squares (ILS), principal-component regression (PCR), artificial neural network (ANN), partial least-squares (PLS) and net-analyte signal (NAS). When a spectrometer other than the cited FTIR spectrometer is used, the spectra are different, but the same chemometrics methods can be used.

In step 140, the at least two compositional variable parameters are analyzed by chemometrics software, which by correlation identifies at least one property or composition of the sample. The at least one property or composition can include density, with values comparable to those that could conventionally be determined by the ASTM D4052 method; refractive index, with values comparable to those that could conventionally be determined by the ASTM D1218 method; viscosity, with values comparable to those that could conventionally be determined by the ASTM D445 method; hydrogen and/or carbon content, with values comparable to those that could conventionally be determined by the ASTM D5291 method; cetane number, with values comparable to those that could conventionally be determined by the ASTM D613 method; cetane index, with values comparable to those that could conventionally be determined by the ASTM D973 method; distillation, with values comparable to those that could conventionally be determined by the ASTM D2887 method; octane number, with values comparable to those that could conventionally be determined by the ASTM D2699 or D2700 methods; cloud point, with values comparable to those that could conventionally be determined by the ASTM D2500 method; pour point, with values comparable to those that could conventionally be determined by the ASTM D97 method; and aniline point with values comparable to those that could conventionally be determined by the ASTM D611 method.

In addition, where the at least two compositional variable parameters are paraffins and naphthenes, the aromatics can be identified in step 140. Similarly, where the at least two composition variable parameters are paraffins and aromatics, the naphthenes can be identified in step 140. Similarly, where the at least two composition variable parameters are naphthenes and aromatics, the paraffins can be identified in step 140. For each of these, the values identified would be comparable to those otherwise determinable by the ASTM D2425 method. The properties identified in step 140 are stored into the non-volatile memory device.

The distillate fractions can be derived from raw or processed petroleum, coal, coal liquid, biomaterials, or synthetic crude oils.

In a preferred embodiment, the composition and/or property determinations are carried out at ambient temperatures and pressures, such as 20° C. and 1 bar.

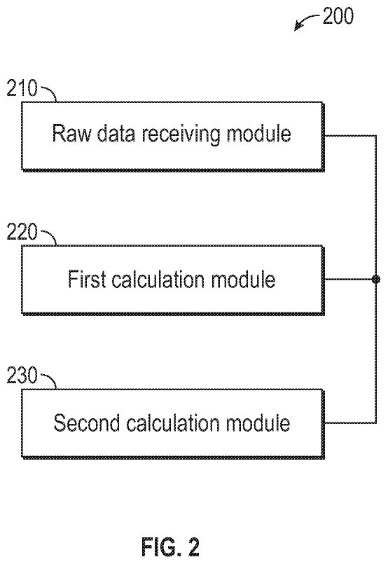

FIG. 2 illustrates a schematic block diagram of modules in accordance with an embodiment of the present invention, system 200. Raw data receiving module 210 receives the spectroscopic data derived from the spectroscopic analysis of the sample of the distillate, and saves the spectroscopic data into the non-volatile memory device.

First software program module 220 receives the spectroscopic data as input, and predicts values of at least two of the compositional variable parameters of paraffins, naphthenes, aromatics. These predicted values of the at least two compositional variable parameters are stored into the non-volatile memory device.

Second software program module 230 receives the at least two compositional variable parameters, and applies chemometrics to predict, by correlation, values of at least one property or composition of the distillate, such as discussed above. The predicted values of the at least one property or properties identified are stored into the non-volatile memory device.

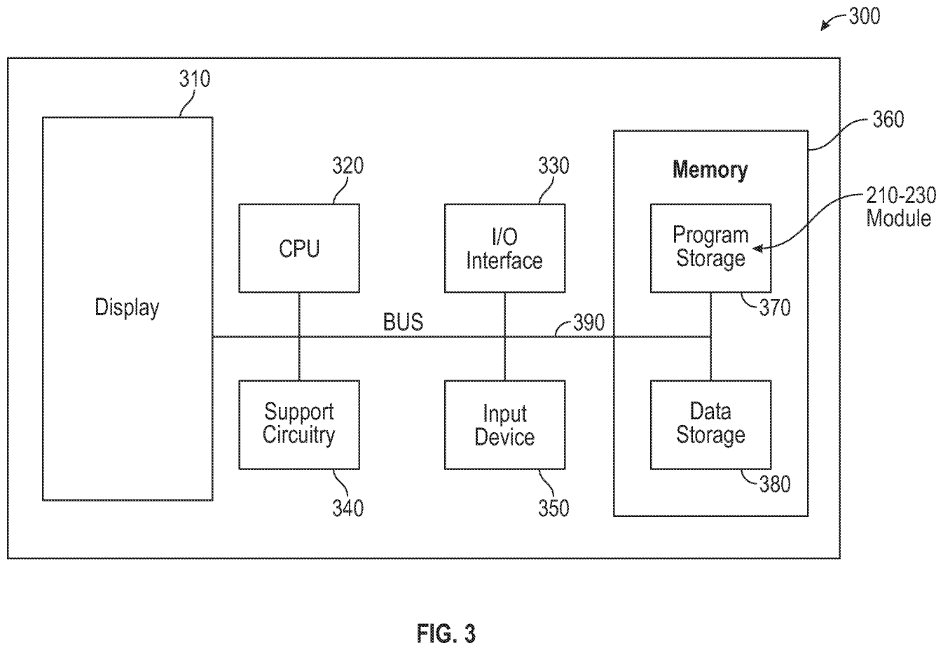

FIG. 3 shows an exemplary block diagram of a computer system 300 in which one embodiment of the present invention can be implemented. Computer system 300 includes a processor 320, such as a central processing unit (CPU), an input/output interface 330 and support circuitry 340. In certain embodiments, where the computer system 300 requires a direct human interface, a display 310 and an input device 350 such as a keyboard, mouse or pointer are also provided. The display 310, input device 350, processor 320, and support circuitry 340 are shown connected to a bus 390 which also connects to a memory 360. Memory 360 includes program storage memory 370 and data storage memory 380. Note that while computer system 300 is depicted with direct human interface components display 310 and input device 350, programming of modules and exportation of data can alternatively be accomplished over the input/output interface 330, for instance, where the computer system 300 is connected to a network and the programming and display operations occur on another associated computer, or via a detachable input device as is known with respect to interfacing programmable logic controllers.

Program storage memory 370 and data storage memory 380 can each comprise volatile (RAM) and non-volatile (ROM) memory units and can also comprise hard disk and backup storage capacity, and both program storage memory 370 and data storage memory 380 can be embodied in a single memory device or separated in plural memory devices. Program storage memory 370 stores software program modules and associated data, and in particular stores the raw data receiving module 210, first software program module 220, and second software program module 230. Data storage memory 380 stores results and other data generated by the software program modules of the present invention.

It is to be appreciated that the computer system 300 can be any computer such as a personal computer, minicomputer, workstation, mainframe, a dedicated controller such as a programmable logic controller, a tablet or smart phone, or a combination thereof. While the computer system 300 is shown, for illustration purposes, as a single computer unit, the system can comprise a group of computers which can be scaled depending on the processing load and database size.

Computer system 300 preferably supports an operating system, for example stored in program storage memory 370 and executed by the processor 320 from volatile memory. According to an embodiment of the invention, the operating system contains instructions for interfacing computer system 300 to the Internet and/or to private networks.

In alternate embodiments, the present invention can be implemented as a computer program product for use with a computerized computing system. Those skilled in the art will readily appreciate that programs defining the functions of the present invention can be written in any appropriate programming language and delivered to a computer in any form, including but not limited to: (a) information permanently stored on non-writeable storage media (e.g., read-only memory devices such as ROMs or CD-ROM disks); (b) information alterably stored on writeable storage media (e.g., floppy disks and hard drives); and/or (c) information conveyed to a computer through communication media, such as a local area network, a telephone network, or a public network such as the Internet. When carrying computer readable instructions that implement the present invention methods, such computer readable media represent alternate embodiments of the present invention.

As generally illustrated herein, the system embodiments can incorporate a variety of computer readable media that comprise a computer usable medium having computer readable code means embodied therein. One skilled in the art will recognize that the software associated with the various processes described can be embodied in a wide variety of computer accessible media from which the software is loaded and activated. Pursuant to In re Beauregard, 35 U.S.P.Q.2d 1383 (U.S. Pat. No. 5,710,578), the present invention contemplates and includes this type of computer readable media within the scope of the invention. In certain embodiments, pursuant to In re Nuijten, 500 F.3d 1346 (Fed. Cir. 2007) (U.S. patent application Ser. No. 09/211,928), the scope of the present claims is limited to computer readable media, wherein the media is both tangible and non-transitory.

The system and method of the present invention have been described above and with reference to the attached figures; however, modifications will be apparent to those of ordinary skill in the art and the scope of protection for the invention is to be defined by the claims that follow.

1. A system for evaluating a sample of a distillate fraction and determining at least one property or composition of the distillate fraction, the system comprising:
a spectrometer that performs a spectrographic analysis of the sample, the spectrometer being a selected one of (i) a near infrared (NIR) spectrometer, (ii) a Fourier transform infrared (FTIR) spectrometer, (iii) a nuclear magnetic resonance (NMR) spectrometer, (iv) an ultraviolet visible (UV-Vis) spectrometer, (v) a Fourier transform-ion cyclotron-mass spectrometer (FT-ICR MS), (vi) a time-of-flight mass spectrometer (TOF-MS), (vii) a laser inducted UV spectrometer, and (viii) a fluorescence spectrometer;
a non-volatile memory device that stores software program modules and data, the data including spectroscopic data as derived by the analysis of the sample by the spectrometer;
a processor coupled to the non-volatile memory device;
a first software program module that is stored in the non-volatile memory device and that is executed by the processor, the first software module predicting values of at least two compositional variables of the sample from the spectroscopic data, and storing the predicted values of the compositional variables into the non-volatile memory device, wherein the at least two compositional variables are selected from (i) paraffins, (ii) naphthenes, and (iii) aromatics;
a second software program module that is stored in the non-volatile memory device and that is executed by the processor, the second software program module using chemometrics to analyze the at least two compositional variables predicted by the first software program module and to predict a value by correlation of the at least one property or composition, wherein the at least one property is selected of (i) density, (ii) refractive index, (iii) viscosity at 40° C., (iv) cetane number, (v) cetane index, (vi) distillation, (vii) octane number, (viii) cloud point, (ix) pour point, and (x) aniline point, and wherein the at least one composition is selected of (i) hydrogen, (ii) carbon, (iii) paraffins, (iv) naphthenes, and (v) aromatics; and wherein the second software program module will store the predicted value of the at least one property or composition into the non-volatile memory device.

2. The system of claim 1, wherein the spectrometer is a near infrared (NIR) spectrometer, and the wavenumber of the spectrum is in the range of 4,000-12,821 cm⁻¹.

3. The system of claim 1, wherein the spectrometer is a Fourier transform infrared (FTIR) spectrometer, and the wavelength is in the range of 650-2000 cm⁻¹.

4. The system of claim 1, wherein the spectrometer is a nuclear magnetic resonance (NMR) spectrometer, that is carbon- or hydrogen-based.

5. The system of claim 1, wherein the spectrometer is an ultraviolet visible (UV-Vis) spectrometer, and the wavelength is in the range of 220-900 nm.

6. The system of claim 1, wherein the spectrometer is a Fourier transform-ion cyclotron-mass spectrometer (FT-ICR MS), covering masses in the range of 150-1400 m/z.

7. The system of claim 1, wherein the spectrometer is a Fourier transform-ion cyclotron-mass spectrometer (FT-ICR MS), covering carbon numbers in the range 1-60.

8. The system of claim 1, wherein the spectrometer is a time-of-flight mass spectrometer (TOF-MS), operating at 5-20 kHz repetition rates.

9. The system of claim 1, wherein the spectrometer is a fluorescence spectrometer, with a wavelength in the range of 250-800 nm.

10. The system of claim 1, wherein the distillate fraction is a selected one of (i) raw petroleum, (ii) processed petroleum, (iii) coal, (iv) coal liquid, (v) biomaterials, and (vi) synthetic crude oil.

11. A method for evaluating a sample of a distillate fraction and determining at least one property or composition of the distillate fraction, the method comprising:
providing a spectrometer, being a selected one of (i) a near infrared (NIR) spectrometer, (ii) a Fourier transform infrared (FTIR) spectrometer, (iii) a nuclear magnetic resonance (NMR) spectrometer, (iv) an ultraviolet visible (UV-Vis) spectrometer, (v) a Fourier transform-ion cyclotron-mass spectrometer (FT-ICR MS), (vi) a time-of-flight mass spectrometer (TOF-MS), (vii) a laser inducted UV spectrometer, and (viii) a fluorescence spectrometer;
providing a non-volatile memory device that stores software program modules and data, including at least a first software program module and a second software program module;
providing a processor coupled to the non-volatile memory device;
conducing a spectrographic analysis of the sample by the spectrometer;
executing the first software program module by the processor to predict values of at least two compositional variables of the sample from the spectroscopic data, and storing the predicted values of the at least two compositional variables into the non-volatile memory device, wherein the at least two compositional variables are selected from (i) paraffins, (ii) naphthenes, and (iii) aromatics;
executing the second software program module by the processor to use chemometrics to analyze the at least two compositional variables predicted by the first software program module and to predict a value by correlation of the at least one property or composition, and storing the predicted value of the at least one property or composition into the non-volatile memory device, wherein the at least one of the properties are selected of (i) density, (ii) refractive index, (iii) viscosity at 40° C., (iv) cetane number, (v) cetane index, (vi) distillation, (vii) octane number, (viii) cloud point, (ix) pour point, and (x) aniline point, and the at least one of the compositions selected of (i) hydrogen, (ii) carbon, (iii) paraffins, (iv) naphthenes, and (v) aromatics.

12. The method of claim 11, wherein the spectrometer is a near infrared (NIR) spectrometer, and the wavenumber of the spectrum is in the range of 4,000-12,821 cm⁻¹.

13. The method of claim 11, wherein the spectrometer is a Fourier transform infrared (FTIR) spectrometer, and the wavelength is in the range of 650-2000 cm⁻¹.

14. The method of claim 11, wherein the spectrometer is a nuclear magnetic resonance (NMR) spectrometer, that is carbon- or hydrogen-based.

15. The method of claim 11, wherein the spectrometer is an ultraviolet visible (UV-Vis) spectrometer, and the wavelength is in the range of 220-900 nm.

16. The method of claim 11, wherein the spectrometer is a Fourier transform-ion cyclotron-mass spectrometer (FT-ICR MS), covering masses in the range of 150-1400 m/z.

17. The method of claim 11, wherein the spectrometer is a Fourier transform-ion cyclotron-mass spectrometer (FT-ICR MS), covering carbon numbers in the range 1-60.

18. The method of claim 11, wherein the spectrometer is a time-of-flight mass spectrometer (TOF-MS), operating at 5-20 kHz repetition rates.

19. The method of claim 11, wherein the spectrometer is a fluorescence spectrometer, with a wavelength in the range of 250-800 nm.

20. The method of claim 11, wherein the distillate fraction is a selected one of (i) raw petroleum, (ii) processed petroleum, (iii) coal, (iv) coal liquid, (v) biomaterials, and (vi) synthetic crude oil.
